(19) United States
(12) Patent Application Publication
(10) Pub. No.: US 2024/0109273 A1
(43) Pub. Date: Apr. 4, 2024

(54) Product for the Storage of Freeze-Dried Lactic Acid Bacteria Mixed with Oral Rehydration Solution

(71) Applicant: BioGaia AB, Stockholm (SE)

(72) Inventor: Christoffer Lundqvist, Lund (SE)

(73) Assignee: BioGaia AB, Stockholm (SE)

(21) Appl. No.: 18/450,498

(22) Filed: Aug. 16, 2023

Related U.S. Application Data
(63) Continuation of application No. 12/803,758, filed on Jul. 6, 2010, now abandoned.
(60) Provisional application No. 61/270,566, filed on Jul. 10, 2009.

(51) Int. Cl. B32B 15/20; A23K 10/18; A23L 33/135; A61K 35/74; B32B 15/085; B32B 27/18; B32B 27/32

(52) U.S. Cl. CPC B32B 15/20; A23K 10/18; A23L 33/135; A61K 35/74; B32B 15/085; B32B 27/18; B32B 27/32; A23V 2002/00; A23V 2400/173; B32B 2307/7246; B32B 2439/00

(57) ABSTRACT

A method of packaging of products that are sensitive to moisture is provided, consequently prolonging the shelf-life of such a product, more specifically prolonging the shelf-life of freeze dried lactic acid bacteria mixed with oral rehydration solution (ORS) powder. The packaging includes two desiccants: one desiccant integrated in the foil material and the second desiccant being the anhydrous ORS.

### CROSS-REFERENCE TO RELATED APPLICATIONS

This application claims priority from U.S. Provisional Application Ser. No. 61/270,566 filed Jul. 10, 2009.

### FIELD OF THE INVENTION

The present invention relates generally to the packaging of products that are sensitive to moisture, consequently prolonging the shelf-life of such a product. More specifically this invention relates to prolonging the shelf-life of freeze dried lactic acid bacteria mixed with an oral rehydration solution (ORS) powder. The product is a package comprising of two desiccants: one desiccant integrated in the foil material and the second desiccant being anhydrous ORS. This invention also relates to methods for production of such products.

### BACKGROUND OF THE INVENTION

Probiotics are live microbial food supplements that beneficially affect the host by improving its intestinal microbial balance. Nowadays, a number of different bacteria are used as probiotics for example, lactic-acid producing bacteria such as strains of Lactobacillus and Bifidobacteria. Lactic-acid producing bacteria are not only used for their beneficial effect on human or animal health, but they are also widely used in the food industry for fermentation processes. Generally, the microorganisms marketed for these purposes are formulated as freeze-dried powders in a low water environment.

A general problem encountered in the application of such lyophilized microorganism preparations is the limited storage stability of the cells. For instance, over time the microorganisms become less viable resulting in high dosages being necessary to compensate for this loss of activity.

It is generally known to utilize aluminum foil as packaging material for the storage of microorganisms, and most preferably foil having a plastic layer on one surface on the outside of the compartment, such as a polyethylene laminated aluminum foil, as packaging material to reduce exposure of freeze dried lactic acid bacteria to moisture and oxygen.

Although the moisture barrier of conventional packages may be useful in restricting the movement of moisture into a package, some moisture molecules can still make their way into the package to deleteriously affect the product contained therein. In addition, even when barrier materials are effective at restricting the transmission of water molecules through a package, certain features of the package may still allow for the transmission of water molecules, for example, along the edges of a heat sealed package. Also, the moment of packaging of the lyophilized microorganisms is a particularly weak point considering moisture absorption.

One solution to maintaining a particularly low or virtually nonexistent level of moisture within a package is to incorporate sachets of desiccant material into the internal space of the package to remove the moisture from the headspace of the package. The desiccant material contained in the sachets is typically in powder or granular form and may leak or otherwise spill from the sachets thereby contaminating the product or products contained within the package. Ingesting desiccants known in the market is not suitable for people suffering from diarrhea, for example. On the contrary it could create deleterious effects. However this issue is solved by the invention herein, wherein the purpose of the desiccant inside the sachet is for it to be consumed.

Typical desiccant materials are “physical” desiccant materials, such as molecular sieves, that bind water molecules within the pore spaces of a material. Typically, physical desiccant materials absorb water at all humidity levels, but will cease to absorb water when the interstices of the physical desiccant material are filled. Therefore, physical desiccant materials may be ineffective at high humidity levels.

Another type of desiccant material is hydrate-forming agents such as salts. Typical salts that may be utilized as desiccant material are magnesium sulfate, sodium phosphate di-basic, ammonium chloride, potassium carbonate, potassium aluminum disulfate, magnesium chloride, diammonium sulfate, sodium nitrate, calcium chloride, and calcium sulfate, although many others are known as well. The drying capacity of these materials is greatly influenced by the relative humidity within a package. Generally, no water is taken up by the hydrate-forming agent until the relative humidity reaches a value at which the first hydrate forms. In the case of calcium chloride, for example, the first hydrate occurs at less than about two percent relative humidity (RH). Water is then taken up by the hydrate forming salt until the first hydrate is completely formed by the salt. No further water is taken up by the salt until the relative humidity reaches a second level where the second hydrate forms. This process continues through as many hydrates as the agent forms, at which point the substance begins to dissolve and a saturated solution is formed. The saturated solution will then continue to take up water.

Although these salts may be effective at removing water molecules from a quantity of gas that may be contained within the headspace of a package, since the salt only binds the water molecules within the salt, the water molecules may easily escape back into the package. This is known as breathing, and may cause deliquescence (water droplets and liquidization) inside the package. Typically, this can happen if the salt becomes saturated and if the temperature of the package increases, or if the pressure of the package decreases, which may occur during shipment or storage of the package.

In addition, salts may not allow moisture levels within a package to fall to a level that is necessary to protect the moisture-sensitive product that may be contained within the package. Typically, since salts have different levels of hydration, humidity levels may remain at a certain level without decreasing until the level of hydration changes. These salts may be utilized to maintain certain humidity levels within the headspace of a package. For example, certain products may require that a certain level of moisture or humidity be maintained within the package headspace. Headspace humidity control for products can be manipulated by incorporation of the appropriate hydrate forming agents.

Desiccant materials may also be used that form no hydrates, such as common salt (NaCl) or potassium bromide (KBr). For example, common salt will absorb no water at a relative humidity below about 75 percent. When 75 percent relative humidity is reached, a saturated solution is formed which continues to take up water.

Another type of desiccant uses chemical desiccant technology. Chemical desiccant materials typically absorb water at all humidity levels, and will continue to take up water at high relative humidity levels. U.S. patent application No. 20070160789 A1 describes a multilayer plastic polymeric flexible packaging foil having the chemical desiccant material incorporated within a layer of the foil. The foil is preferably polyethylene selected from the group consisting of ultra low density polyethylene, low density polyethylene, linear low density polyethylene, medium density polyethylene, and high density polyethylene. This invention utilizes such desiccants as calcium oxide (preferred), magnesium oxide, barium oxide, strontium oxide, aluminum oxide, partially hydrated aluminum oxide, magnesium sulfate, sodium phosphate di-basic, ammonium chloride, potassium carbonate, potassium aluminum disulfate, magnesium chloride, diammonium sulfate, sodium nitrate, calcium chloride, calcium sulfate, sodium chloride, potassium bromide, molecular sieves, clays, or any other desiccant material useful for the present invention.

Oral rehydration therapy (ORT) is a simple, cheap, and effective treatment for dehydration associated with diarrhea. ORT consists of a solution of salts and sugars which are administered orally also called oral rehydration solution (ORS) and oral rehydration formula (ORF). ORT is used around the world, but is most important in the developing world, where it saves millions of children from death due to diarrhea—the second leading cause of death in children under five.

Several trials have been conducted to evaluate the efficacy of lactic-acid bacteria administered in the oral rehydration solution. For example, a multicenter trial was conducted to evaluate the efficacy of Lactobacillus GG administered in the oral rehydration solution to patients with acute-onset diarrhea of all causes, showing that administering oral rehydration solution containing Lactobacillus GG to children with acute diarrhea is safe and results in shorter duration of diarrhea, less chance of a protracted course, and faster discharge from the hospital (J Pediatr Gastroenterol Nutr. 2000 January; 30(1):54-60). However as mentioned before the problem is that freeze dried lactic acid bacteria require a low water activity (usually a_{w}<0.2) in a powder matrix to have a reasonable shelf life at ambient temperature without a dramatic loss in viability.

Due to the extremely hygroscopic (ability to attract water molecules from the surrounding environment) nature of oral rehydration solution powder, it has until now not been possible to mix a freeze dried lactic acid bacteria with an ORS powder and keep the bacteria viable during ambient storage for a longer period of time. The variable storage quality of live probiotic cultures results in unpredictable losses of dose and activity in the preparations administered to patients with acute watery diarrhea, and this is a technologic problem that is difficult to solve, particularly in developing countries.

From the storage point of view, a method was tested in the invention herein to evaluate whether any salt-mixtures could successfully work as desiccant inside a package, mixed with freeze dried Lactobacillus reuteri. The package was made of the foil material described in US20070160789 A1. Surprisingly the freeze-dried cultures of L. reuteri mixed with anhydrous ORS successfully reached at least 12 months of shelf life at 30° C. which in known to mean longer storage times at lower temperatures. Advantageous storage stability data of this type have not been described hitherto. As far as we know there is no such product on the market anywhere in the world.

In summary, it has until now not been possible to mix a freeze dried lactic acid bacteria with an ORS powder and still keep the bacteria viable during ambient storage in any form of package for a longer period of time. The solution to this problem is solved by the invention herein by removing moisture in two steps with two different desiccants. The first desiccant is anhydrous ORS working as a desiccant, surrounding the microorganisms at the moment of packaging, and the secondly desiccant is in the foil absorbing the moisture possibly arising during storage. When anhydrous ORS is used, it functions both as a desiccant that does not need to be removed simultaneously as it fulfills its aim as an oral rehydration solution.

### SUMMARY OF THE INVENTION

The invention herein provides a method of packaging of products that are sensitive to moisture, consequently prolonging the shelf-life of such a product, more specifically prolonging the shelf-life of freeze dried lactic acid bacteria mixed with oral rehydration solution (ORS) powder. The package comprises two desiccants: one desiccant integrated in the foil material and the second desiccant being anhydrous ORS.

A primary object of the present invention is to provide a product prolonging the shelf-life of freeze dried lactic acid bacteria mixed with oral rehydration solution (ORS) powder

Another object of the present invention is to provide a package with two desiccants removing moisture in two steps for the storage of freeze-dried lactic acid bacteria.

Another object is to provide a sachet with two desiccants: one desiccant integrated in the foil material, described in US20070160789 A1, and the other desiccant being anhydrous ORS, present freely inside the sachet, surrounding the product.

Other objects and advantages of the present invention will become obvious to the reader and it is intended that these objects and advantages are within the scope of the present invention.

### BRIEF DESCRIPTION OF THE DRAWINGS

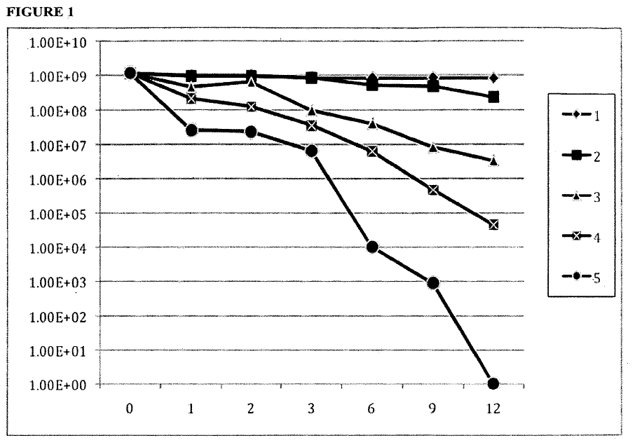

FIG. 1 is a graph showing the viability of Lactobacillus reuteri with different desiccants at +30° C. on a monthly basis up to 12 months:
1) L. reuteri+foil with desiccant
2) L. reuteri+foil with desiccant+anhydrous ORS
3) L. reuteri+foil with desiccant+non-anhydrous ORS
4) L. reuteri+foil without desiccant+anhydrous ORS
5) L. reuteri+foil without desiccant+non-anhydrous ORS

### DETAILED DESCRIPTION OF THE INVENTION AND PREFERRED EMBODIMENTS THEREOF

The method and device of the invention provide packaging of products that are sensitive to moisture, consequently prolonging the shelf-life of such a product, more specifically prolonging the shelf-life of freeze dried lactic acid bacteria mixed with oral rehydration solution (ORS) powder. The package comprises two desiccants: one desiccant integrated in a aluminum foil and the second desiccant being anhydrous ORS. The foil is preferably made of aluminum, with a suitable protective coating such as polyethylene or a lacquer. The reason being is that aluminum foil is bar far the best known barrier against moisture and the products discussed herein are very sensitive to moisture. Testing of the barrier characteristics of the package is done as known in the art (Allinson et al, International Journal of Pharmaceutics Volume 221, Issues 1-2, 19 Jun. 2001, Pages 49-5)

The term “anhydrous ORS” as used herein means that the components of the ORS powder are of anhydrous quality i.e. free of water. The components can be bought in anhydrous form or can be made anhydrous by methods known in the art.

The goal of the invention herein is to provide a method and device that results in survival of the freeze dried lactic acid bacteria such that at the end of 12 months, at storage around +30° C. there are at least 10E+07 CFU/gram (when starting with 5×10E+09 CFU/gram) but preferably at least 10E+08 CFU/gram and best 10E+09 CFU/gram at this temperature and starting point. This stability is known to translate to maximum the same amount of losses at around 18 months when the product is stored in +25° C. and less when the temperature is lower. This would mean that the goal at 18 month at +25° C. there are at least 10E+07 CFU/gram (when starting with 5×10E+09 CFU/gram) but preferably at least 10E+08 CFU/gram and best 10E+09 CFU/gram at this temperature and starting point.

The features of the present invention will be more clearly understood by reference to the following examples, which are not to be construed as limiting the invention.

### Example 1

### Parameters Tested

In the examples herein, the method of the invention is tested to evaluate if ORS solution salts can be mixed with freeze dried L. reuteri in a sachet with the aim of reaching at least 12 months of shelf life at 30° C. as an accelerated study which in known to mean longer storage times at lower temperatures. The new production method includes the use of a new foil material, with a desiccant, described in US20070160789 A1.

The assays undertaken consist of five different test parameters as shown in Table 1:

TABLE 1

| | | | | PET12/PE/ALU 12/ | PETP 12/ALU 9/ |
|---|---|---|---|---|---|
| Test | L. reuteri | ORS | | PE/PE + desiccant/ | LLDPE70 from |
| parameter | DSM 17938 | anhydrous | ORS | PE from Alcan * | Amcor Flexibles ** |
| 1 | X | | | X | |
| 2 | X | X | | X | |
| 3 | X | | X | X | |
| 4 | X | X | | | X |
| 5 | X | | X | | X |

* Foil with integrated desiccant described in US20070160789 A1
** Reference foil without integrated desiccant

### Example 2

Production of Sachets Comprising a Mix of L. reuteri in Anhydrous O.R.S Powder

The anhydrous ORS powder contained:
L. reuteri DSM 17938: 68.000 mg/sachet at around 10E+11 CFU/gram
Glucose anhydrous: 3781.000 mg/sachet
Sodium citrate anhydrous: 866.000 mg/sachet
Potassium chloride: 379.000 mg/sachet
Sodium chloride: 365.000 mg/sachet
Zinc sulphate: 4.000 mg/sachet
TOTAL 5463.000 mg/sachet

The blend was filled at ambient temperature into aluminum foil bags with desiccant (10 cm×12 cm, using packaging material PET12/PE/ALU 12/PE/PE+desiccant/PE from Alcan) in a LAF bench (Holten Laminair Model S-2010 1.2 from Heto-Holten A/S, Denmark) at the microbiological laboratory at BioGaia Lund. To each bag 5.46 g of ORS powder with L. reuteri was added using the balance XP-600 from Denver Instrument GmbH, Germany. The filled aluminum foil bags were then heat sealed with the foil sealing device model F460/2 from Kettenbaum Folienschweisstechnik GmbH & Co. KG, Germany. As there is practically no permeability of moisture at all trough the aluminum foil, especially if the aluminum is thicker than 0.025 mm and therefore most prefered, the seal is the sensitive part of the total enclosure, which means that the sealing device use must be of high quality giving unbroken seals without any leaks and sufficient with as known in the art. These bags were also used in the study described in example 2.

### Example 3

Stability Study of Freeze Dried L. reuteri DSM 17938 During Storage in Oral Rehydration Solution (ORS) Powder at 30° C., During 12 Months

The viability of L. reuteri DSM 17938 powder art no 1012 BioGaia AB, Stockholm, Sweden (product specification PS137) is evaluated in an oral rehydration solution powder from Norfoods AB, Sweden. The anhydrous ORS powder is made according to example 1. The test parameters are performed according to table 1. The samples are stored in climate cabinets at 30° C./65% RH at BioGaia AB in Lund, Sweden.

### Example 4

The results are shown in FIG. 1. The microorganisms stored in the foil with integrated desiccant and anhydrous ORS surprisingly (graph 2 in FIG. 1) showed almost the same viability as the microorganisms stored without ORS (graph 1 in FIG. 1).

The microorganisms stored in the foil with integrated desiccant showed better viability during storage than the microorganisms stored in the foil without desiccant, since the desiccant in the foil absorbs almost all of the moisture within the bag during storage. But only having the foil with desiccant and non-anhydrous ORS (graph 3 in FIG. 1) is shown to not be enough since the viability is poorer than with the microorganisms stored in the foil with integrated desiccant and anhydrous ORS (graph 2 in FIG. 1)

Conclusively, neither the anhydrous ORS alone nor the foil with integrated desiccant alone is enough to provide a product with sufficient shelf-life. Storage including both of them together fulfills the activity goal at the end of shelf life.

While the invention has been described with reference to specific embodiments, it will be appreciated that numerous variations, modifications, and embodiments are possible, and accordingly, all such variations, modifications, and embodiments are to be regarded as being within the spirit and scope of the invention.

1. A probiotic product with prolonged shelf-life, comprising: freeze-dried Lactobacillus reuteri DSM 17938 mixed with anhydrous oral rehydration solution powder, wherein the Lactobacillus reuteri DSM 17938 and the anhydrous oral rehydration solution powder are packaged in a foil having a chemical desiccant integrated in the foil, and wherein the anhydrous oral rehydration solution powder comprises an anhydrous sugar; anhydrous salt comprising one or more of sodium, chloride, potassium, bicarbonate or citrate; and at least one mineral.

2. The probiotic product of claim 1, wherein the foil comprises aluminum.

3. The probiotic product of claim 2, wherein the foil further comprises a polyethylene layer.

4. The probiotic product of claim 1, wherein the desiccant integrated in the foil material comprises calcium oxide.

5. (canceled)

6. The probiotic product of claim 1, wherein after 12 months of storage of the probiotic product at 30° C. there are at least 10E+07 CFU/gram of Lactobacillus reuteri DSM 17938, when starting with 5×10E+09 CFU/gram of Lactobacillus reuteri DSM 17938.

7. The probiotic product of claim 6, wherein there are at least 10E+08 CFU/gram of Lactobacillus reuteri DSM 17938 after 12 months of storage.

8. The probiotic product of claim 7, wherein there are 10E+09 CFU/gram of Lactobacillus reuteri DSM 17938 after 12 months of storage.

9.-12. (canceled)

13. A method of prolonging the shelf-life and long-term storage of viable freeze-dried Lactobacillus reuteri DSM 17938 bacteria, comprising:
a) mixing freeze-dried Lactobacillus reuteri DSM 17938 with an anhydrous oral rehydration solution powder comprising an anhydrous sugar; anhydrous salt comprising one or more of sodium, chloride, potassium, bicarbonate or citrate; and at least one mineral;
b) packaging the mixed freeze-dried Lactobacillus reuteri DSM 17938 and oral rehydration solution powder in a foil having a chemical desiccant integrated in the foil; and
c) sealing the packages so that the seal is unbroken.

14. The method of claim 13, wherein the foil comprises aluminum.

15. The method of claim 14, wherein the foil further comprises a polyethylene layer.

16. The method of claim 13, wherein the desiccant integrated in the foil material comprises calcium oxide.

17. (canceled)

18. The probiotic product of claim 1, wherein the anhydrous sugar comprises anhydrous glucose or anhydrous dextrose.

19. The probiotic product of claim 1, wherein the anhydrous oral rehydration solution powder comprises anhydrous sodium citrate.

20. The probiotic product of claim 1, wherein the anhydrous oral rehydration solution powder comprises an anhydrous sugar, anhydrous sodium citrate, potassium chloride, sodium chloride, and zinc sulfate.

21. The probiotic product of claim 1, wherein the anhydrous oral rehydration solution powder comprises anhydrous glucose or dextrose, anhydrous sodium citrate, potassium chloride, sodium chloride, and zinc sulfate.

22. The probiotic product of claim 1, wherein the anhydrous oral rehydration solution powder consists essentially of anhydrous glucose or dextrose, anhydrous sodium citrate, potassium chloride, sodium chloride, and zinc sulfate.

23. The probiotic product of claim 1, wherein the freeze-dried Lactobacillus reuteri DSM 17938 in the probiotic product remain viable for a longer period of time when stored at the same temperature than freeze-dried bacteria (a) mixed with non-anhydrous oral rehydration powder and packaged without desiccant, (b) mixed with anhydrous oral rehydration powder and packaged without desiccant, and (c) mixed with non-anhydrous oral rehydration powder and packaged with desiccant.

24. A method of treating diarrhea in a patient, comprising administering to the patient an effective amount of the probiotic product of claim 1.
